(19) United States
(12) Patent Application Publication
(10) Pub. No.: US 2022/0133583 A1
(43) Pub. Date: May 5, 2022

(54) SKIN TREATMENT DEVICE

(71) Applicant: NSE Products, Inc., Provo, UT (US)

(72) Inventors: Richard Best, Mapleton, UT (US); Mukul Bokil, American Fork, UT (US)

(21) Appl. No.: 17/504,876

(22) Filed: Oct. 19, 2021

Related U.S. Application Data
(60) Provisional application No. 63/108,805, filed on Nov. 2, 2020.

(51) Int. Cl. A61H 15/02; A61N 1/32; A61N 1/04; A61N 1/36; A61H 15/00; A61N 1/02; A61M 35/00

(52) U.S. Cl. CPC A61H 15/02; A61N 1/328; A61N 1/0476; A61N 1/36034; A61H 15/0092; A61N 1/36031; A61H 2201/1692; A61M 35/003; A61H 2015/0014; A61H 2201/105; A61H 2201/5084; A61H 2201/5082; A61N 1/025

(57) ABSTRACT

A skin treatment device includes a roller disposed about a rotational axis, with a plurality of flexible, compressive skin contact elements distributed circumferentially about the roller. Compressive elements include a first longitudinal section coupled to the roller, a second longitudinal section with a radially outer skin contact surface extending opposite the first longitudinal section, and a transverse section with one or more web members connecting the first and second longitudinal sections at an angle. The angles of the web members are adapted to generate lateral displacement of the contact surfaces in response to a compressive load. The lateral displacements of adjacent pairs of contact surfaces are defined in opposite directions, parallel and antiparallel to the rotational axis, in order to provide an elastic stretching treatment to the skin. Electrodes can be disposed adjacent the roller, on the housing or between adjacent contact surfaces, in order to provide a current treatment.

### CROSS-REFERENCE TO RELATED APPLICATION

This application claims priority to U.S. Provisional Application No. 63/108,805, “Skin Treatment Device,” filed Nov. 2, 2020, which is incorporated by reference herein, in the entirety and for all purposes.

### FIELD

This disclosure relates to skin treatment techniques, including roller devices adapted for physical skin stimulation. More generally, the disclosure relates to roller devices adapted for a beneficial, elastic skin stretching treatment, or for micro-current based skin treatment, or a combination thereof.

### BACKGROUND

The skin is the largest organ of the human body, forming a physical barrier to the environment and providing important functions including insulation, temperature regulation and protection against microorganisms, as well as touch, heat sensitivity, and other forms of sensation. The skin also regulates the passage of water and electrolytes, and produces vitamin D.

The outermost skin layer or epidermis covers the body's surface. Most of the epidermal cells are keratinocytes, which form an environmental barrier and synthesize vitamin D. The epidermis also includes melanocytes, which produce melanin to protect against harmful UV radiation, Merkel cells, which provide sensitivity to touch, and Langerhans cells, a type of white blood cell or macrophage that is part of the immune system, acting to protect the body against infection.

The epidermis surrounds the dermis. The structure of the dermis is provided by fibroblasts, which synthesize collagen and elastin proteins to form the extracellular matrix, with collagen fibers to provide strength and toughness, and elastin threads or filaments to provide elasticity and flexibility. The fibroblasts also produce proteoglycans, viscous proteins that provide hydration and lubrication, and regulate ionic binding and molecular transport. The dermis also includes macrophages and mast cells, part of the immune system, as well as the hair follicles, sweat and oil glands, nerve cells, and blood vessels.

The epidermis and dermis make up the cutis. Subcutaneous tissue connects the cutis to the underlying muscle and fascia, and to other connective tissue including the periosteum (covering the bones). The subcutis also includes elastin and adipose (fat) cells.

As the skin ages, loss of firmness and elasticity may be associated with a decrease in the production of Type I collagen (the most abundant form), as well as a reduction in elastin, proteoglycans, and other components of the extracellular matrix. Particularly in certain areas of the human body, a condition called cellulite may result. Aging skin can also exhibit thinning, coloration, and reduced immune response.

A range of products have been provided to help improve skin condition and appearance, including topical products and hand-held devices for cleansing, exfoliating and smoothing the outer skin layers. Known techniques include both mechanical treatment devices adapted to stretch and clean the skin tissues, and galvanic or micro-current based devices designed for electrical stimulation, for example as described in U.S. Pat. Nos. 10,046,160 B1, 10,080,428 B2, 10,661,072 B2, 10,765,199 B2, and 10,772,473 B2, each of which is assigned to NSE (Nu Skin Enterprises) Products, Inc., of Provo Utah, and incorporated by reference herein.

More generally, the skin's response to physical stimulation involves a number of complex and interacting biological processes, and the full range of different treatment mechanisms have not all been recognized in the prior art. As a result, there is an ongoing need for more progressive approaches to skin care, including physical skin stimulation techniques developed with a better understanding of the underlying biological responses, and providing a more favorable treatment response.

### SUMMARY

A skin treatment device is described, along with associated methods of operation. In one example, the device includes a roller disposed along a rotational axis, with a plurality of flexible skin contact or movement elements distributed circumferentially about the roller. The flexible skin contact elements can include a first longitudinal portion coupled to the roller, a second longitudinal portion with a radially outer skin contact surface opposite the first longitudinal portion, and a transverse web portion connecting the first and second longitudinal portions, configured for lateral displacement of the radially outer skin contact surface responsive to a compressive load.

The lateral displacements of adjacent skin contact elements can be defined in opposing directions, in order to provide an elastic stretching treatment. Electrodes can be disposed on a housing adjacent the roller, or distributed between adjacent skin contact elements, in order to provide a beneficial current treatment, for example a microcurrent treatment, or alternatively a galvanic treatment. Additional advantages and features of these embodiments are set forth in the description that follows, and will be apparent to those skilled in the art upon examination of the specification, drawings and claims.

### BRIEF DESCRIPTION OF THE DRAWINGS

FIG. 1 is a side elevation view of a skin treatment device with roller mechanism adapted for application to a subject's skin.

FIG. 2 is an isometric bottom view of the skin treatment device, showing the roller mechanism and skin treatment surface.

FIG. 3A is a top plan view of the skin treatment device, showing the user interface.

FIG. 3B is a bottom plan view of the skin treatment device, showing the skin treatment surface.

FIG. 4 is an alternate bottom view of the skin treatment device, in an alternative two-electrode configuration.

FIG. 5 is a side section view of the skin treatment device, showing the roller in contact with a subject's skin surface.

FIG. 6 is an isometric view of a roller for the skin treatment device, showing the flexible skin contact elements and endcaps.

FIG. 7A is a side section view of the roller, showing the flexible skin contact elements extending radially around an axle.

FIG. 7B is an alternate side section view of the roller, in an embodiment with bar electrodes disposed between the skin contact elements.

FIG. 8A is a side elevation view of a flexible skin contact element for the roller.

FIG. 8B is a side elevation view illustrating compression of the skin contact element, upon application to a subject's skin.

FIG. 8C is a side elevation view of an alternate skin contact element for the roller.

FIG. 9A is a plot of horizontal travel distance versus vertical displacement for representative skin contact elements.

FIG. 9B is a plot of reaction force versus vertical displacement for the representative skin contact elements.

FIG. 10A is a schematic diagram illustrating a stretching effect of the skin contact elements, in operation of the roller.

FIG. 10B is a schematic diagram illustrating distribution of a topical product by a skin contact element, in operation of the roller during displacement.

FIG. 11A is an isometric view of a skin treatment device with the roller supported in an ergonomic handle, and electrical contacts for applying a current treatment to contacted skin.

FIG. 11B is an isometric view of a roller for the skin treatment device of FIG. 11A, showing the electrical contacts.

FIG. 12A is an isometric view of a skin treatment device with the roller supported in an active ergonomic handle.

FIG. 12B is a detail view of a roller endcap for the skin treatment device of FIG. 12A.

FIG. 13 is a block diagram illustrating a skin treatment device.

FIG. 14 is a block diagram illustrating a representative method of using the skin treatment device.

### DETAILED DESCRIPTION

Although the present disclosure describes particular examples and preferred embodiments of the invention, persons skilled in the art will recognize that changes may be made in form and detail without departing from the scope of the claims. The various examples and embodiments are also described with reference to the drawings, where like reference numerals represent similar structural and functional components throughout the several views. These examples and embodiments do not limit practice of the invention as claimed; rather, the specification merely sets forth representative applications to different systems, methods and devices, and practice of the invention is not limited except as set forth in the appended claims.

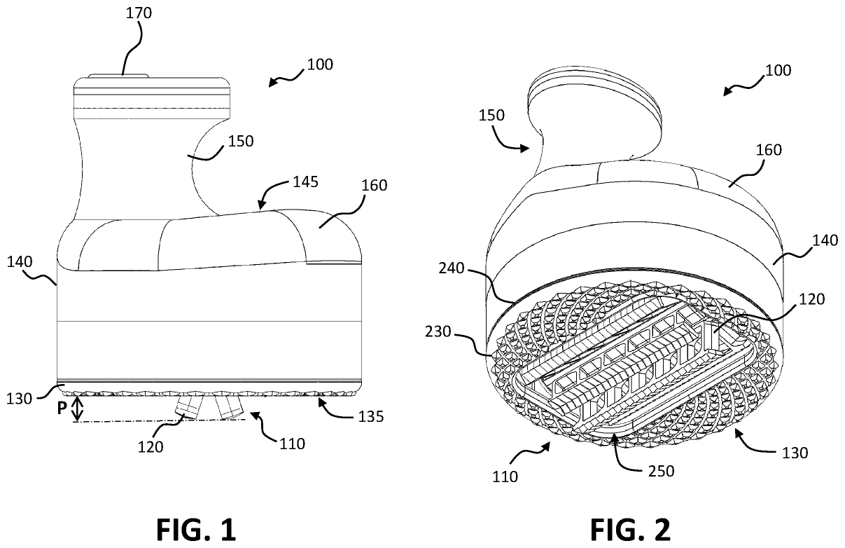

FIG. 1 is a side elevation view of a representative skin treatment device 100, with a roller or rotor mechanism 110 adapted for application to a subject's skin. As shown in FIG. 1, the roller 110 includes a number of flexible, compressive skin contact elements or “skids” 120 extending from an aperture in the bottom surface 130 of housing 140, opposite the top 145 of the device housing 100, and adjacent the skin treatment electrode 135 on bottom surface 130.

In this particular example, device housing 140 has an ergonomic design, with a manual interface, handle or grip 150 on the top housing 145, adapted for user manipulation of device 100. The roller 110 is configured to cleanse the subject's skin, to improve topical distribution and delivery, and to provide an elastic stretching treatment via the operation of the flexible, compressive skin contact elements 120. Such treatments can have a range of beneficial effects, including exfoliation, skin cleaning, and improved skin appearance, and have also been associated with other potential benefits, including enhanced skin elasticity, reduction of undesirable conditions such as cellulite, and improved health of the extracellular matrix (e.g., depending on selected topical treatment, and based on physiological response to the stretching action, at the cellular and molecular level).

The extracellular matrix (ECM) is composed of collagen fibers, elastin fibers, and the water-holding molecules retained within the network of the fibers, for example other proteins and glycosaminoglycans such as chondroitin, biglycan, hyaluronic acid, and the like. Restoring the ECM results in an improvement in appearance and a decrease in the apparent age of the subject. Without limitation, a specific degree, frequency, and period of controlled stretching of human skin, or any combination of two or more such effects, can also result in or relate to additional effects such as micro-extracellular matrix stretching that in turn causes or relates to stretching of the attached dermal fibroblasts. Such stretching can also cause or relate to beneficial changes in gene expression, for example in the fibroblasts or other skin tissues (or both), allowing the body to build, augment or repair components of the extracellular matrix, improving skin health and appearance.

Device 100 can also be adapted to deliver a therapeutic current treatment to the subject's skin, for example by applying a controlled electrical signal or microcurrent waveform (e.g., a voltage or current waveform), via a conducting surface or electrode 135 on the bottom surface (or bottom) 130 of the housing, with current returning through one or more return electrodes 160. The current path can also include a topical agent applied on the skin, enhancing beneficial current flow. For example, the current can be transmitted through a conducting topical agent or treatment fluid applied between the electrode surface 135 and the user or subject's skin. The topical agent can also include cleansing agents, moisturizers or beneficial skin nutrients, or a combination thereof.

The current flowing into the skin can return to device 100 via a path extending through the user's body to one or more current return electrodes (or electrode surfaces) 160 on the housing 140. The housing 140 can be ergonomically designed for coupling with the user's hand (e.g., the palm, heel, and/or fingers) holding on to the handle or grip 150. Alternatively, a therapeutic current treatment can also be applied at skin contacting electrodes placed on roller 110; e.g., a microcurrent treatment applied between adjacent elements 120, or a galvanic treatment, as described herein.

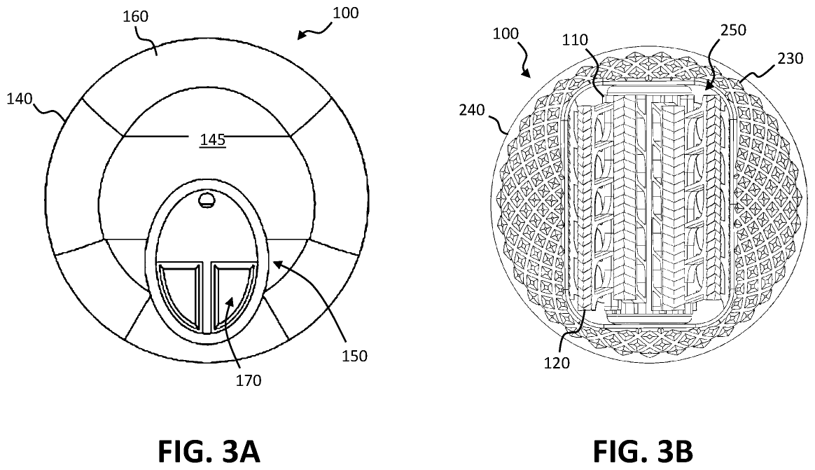

A user display or graphical user interface (GUI) 170 can also be provided on the housing 140, for example on the top of a handle or manual grip 150 as shown in FIG. 3A (described below). Alternatively, a suitable user interface (IF) 170 can be provided on the front or side of housing 140, or in another convenient location, visible and accessible to a user holding the device 100. User interface 170 can be adapted to provide user information such as roller speed, current treatment status, a timer and other operational information, and to accept user input, for example using an on/off control, current level, voltage level, voltage or current waveform, or other operational parameter, as well as other input and output features as described herein, including the incorporated reference.

FIG. 2 is an isometric bottom view of the skin treatment device 100, showing a portion of the roller 110 including two adjacent contact elements 120, and patterned electrode or treatment surface 135. Roller 110 is disposed in roller aperture 250 (e.g., generally oblong or rectangular, in this embodiment), forming the inner perimeter of electrode surface 135 on the bottom of device 100. Roller 110 includes a plurality of flexible, compressive contact elements 120, some of which extend from roller aperture 250 below electrode surface 135, depending on the rotational position of roller 110. Flexible contact elements 120 are thus sequentially applied to the subject's skin though operation of roller 110, in order to provide a therapeutic, elastic stretching treatment as described herein.

In some designs, the extension distance P of contact elements 120 below the patterned electrode surface 135 defines a maximum vertical displacement of the contact elements 120, for example from about 0.5 mm to about 3.5 mm or more. The vertical position of roller 110 within aperture 250 and the extension distance P can also vary, for example from about 1.0 mm to about 5.0 mm or more, or up to 5.0-10.0 mm or more.

In the example of FIGS. 1 and 2, the patterned electrode surface 135 is provided with an interlocking spiral pattern of generally diamond-shaped surface features 230, extending circumferentially about roller aperture 250 to the outer perimeter 240 of electrode surface 135. The patterned surface features 230 can be adapted to help distribute a topical agent or treatment formula over the subject's skin, and to help provide a distributed electrode pattern for the application of a therapeutic current treatment. Depending on application, surface features 230 can also be formed as circular, oval, triangular, square, oblong, ridged, or other (e.g., irregularly shaped) features, or a combination of such features. Surface features 230 can also be provided in a symmetric grid or radial pattern, or in an asymmetric, irregular, or randomized pattern, or other suitable arrangement. The surface pattern can be relatively shallow, with features 230 having a tip to trough dimension (or height, defined at the electrode surface 135) of about 1-2 mm or less, or about 0.5-1.0 mm or less, and a lateral dimension (width, diameter, or length, defined along the electrode surface 135) of 1-2 mm or more, or up to 5 mm or more. Alternatively, the shape and dimensions of surface features 230 may vary from these ranges.

FIG. 3A is a top plan view of skin treatment device 100. As shown in FIG. 3A, housing 140 has an ergonomic design that is adapted to fit the user's hand, for example with a handle or grip 150 disposed between adjacent fingers (or between a thumb and an adjacent finger), and the user's palm against the top 145 of the housing 140, with the palm or heel in contact with one or more return electrodes 160. Electrodes 160 are positioned and dimensioned to distribute the return current over the user's hand, or portion of the body in contact with the top 145 of the housing 140, spaced from the bottom surface 130, reducing current density below a nominal threshold or minimal level selected to improve user comfort.

User interface 170 is adapted to provide operational information to the user, and to receive control commands. Typically, user interface 170 can be configured to provide on/off controls for a therapeutic current treatment, or to select a particular current or microcurrent cycle, a voltage or current level, or a microcurrent waveform type, to control the current density (e.g., based on user comfort), or other operational parameters such as treatment period. Depending on embodiment, user interface 170 can include a smart screen display to provide additional operational information such as remaining treatment duration, treatment history, or user information such as skin resistivity or an elasticity measure, and sensor information provided by humidity, pressure or other sensors embedded in the device 100; e.g., on the patterned electrode or treatment surface 135, or by analysis of operational parameters such as applied voltage and current flow.

In some designs, user interface 170 also provides operational information such as the translational speed of the device 100 across the subject's skin, or the rotational speed of the roller, in order to help user maintain a preselected speed range for the stretching treatment. Suitable sensors can be provided for measuring the speed; e.g., an accelerometer or velocity sensor disposed within or on housing 140, or a rotational sensor coupled to the roller 110, and configured to determine the linear speed of the device 100 across the subject's skin based on the rotational speed of the roller 110 (e.g., using an encoder or resolver, or a hall effect sensor). In these examples, user interface 170 can also be provided with visual, audio or haptic cues to signal whether the speed is over, under, or within the selected range.

The cues can be provided on the device itself, e.g., via visual or audio cues on or from the user interface 170, via haptic feedback (e.g., a vibrator coupled to the device housing 140, guiding the user to move the device 100 at a selected speed across the skin (or within the selected speed range), in order to improve or optimize the skin treatment. Similar cues can also be responsive to the therapeutic current or microcurrent treatment, for example to indicate when the treatment is active, or when the treatment has terminated. Depending on application, some or all display and control functionality of user interface 170 can also be provided via a wired or wireless connection to a mobile device, for example a smart phone, tablet, or other computer device running an application-specific program for user control of device 100, and for tracking usage information.

FIG. 3B is a bottom plan view of the skin treatment device 100, showing conductive, patterned electrode surface 135. Electrode surface 135 can formed with an interlocking spiral pattern of generally diamond-shaped treatment surface features 230, or other suitable arrangement of surface features 230 adapted to distribute a conductive topical agent to the subject's skin, and deliver a therapeutic current or microcurrent treatment.

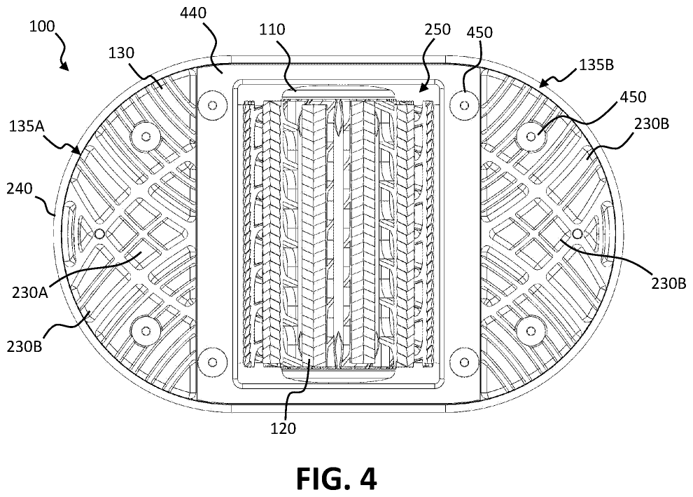

As the user manipulates device 100 along a desired (to-be-treated) skin surface, roller 110 rotates within housing 140, and flexible contact elements 120 sequentially extend though roller aperture 250 in the bottom 130 of housing 140. As the elements 120 contact the subject's skin, they compress in response to the normal force acting back on the radially outer contact surfaces. Upon compression, pairs of adjacent contact elements 120 are adapted to displace in opposite directions along (parallel or antiparallel) to the rotational (longitudinal) axis of roller 110, while the skin contacting surfaces (diamond-shaped or elongated rectangles as seen in the embodiment of FIG. 4), are gently gripping and thereby elastically stretching the subject's skin. The forces applied to displace and stretch the skin with the flexible elements 120 can thus be defined by a pair of vectors oriented in opposing directions, generally parallel (or anti-parallel) to the axis of the roller. This displacement also helps to distribute any topical agent on the skin contacted by the contact elements 120, in order to improve the current path and deliver any moisturizers and nutrients or other substances in the topical agent.

Treatment surface features 230 of electrodes are also adapted to distribute the topical agent over the subject's skin, and to deliver a current or microcurrent treatment. Suitable control signals and waveforms can be selected to regulate the current in order to promote user comfort, ion transport and other biological effects; e.g., as further described in U.S. Publication No. 2007/0185431 A1 and U.S. Publication No. 2021/0162212 A1, in U.S. Pat. Nos. 10,046,160 B1, 10,080,428 B2, 10,661,072 B2, 10,765,199 B2, and 10,772,473 B2, and in U.S. patent application Ser. No. 17/221,415, filed Apr. 2, 2021, to NSE Products, Inc. of Provo, Utah, each of which is incorporated by reference herein, in the entirety and for all purposes.

FIG. 4 is an alternate bottom view of the skin treatment device 100, in a configuration with two separate patterned treatment or electrode surfaces 135A, 135B. In this particular example, the patterned surfaces form two electrodes 135A, 135B, arranged on opposing (e.g., front and back) sides of roller 110.

Roller 110 is disposed within a roller housing 440, which defines an aperture 250 in the bottom surface 130 of the device housing. Roller housing 440 can be mechanically coupled to the housing, for example using one or more screws, bolts, pins, or mechanical fasteners 450 extending through roller housing 440 on the bottom surface 130 of the device. Electrode surfaces 135A, 135B are arranged between roller aperture 250 and the outer perimeter 240 of bottom surface 130, and coupled to bottom surface 130 of device 100 with additional mechanical fasteners 450.

In the embodiment of FIG. 4, electrode surfaces 135A, 135B are provided with a pattern of rectangular, ridged, or oblong surface features 230A and 230B, which are adapted to generate a beneficial microcurrent flow path across tissue beneath roller 110 between (e.g., front and rear) electrode surfaces 135A, 135B, and for distribution of a topical treatment over the subject's skin. The electrode surfaces 135A, 135B may also have a smooth pattern, or be provided with different patterned surface.

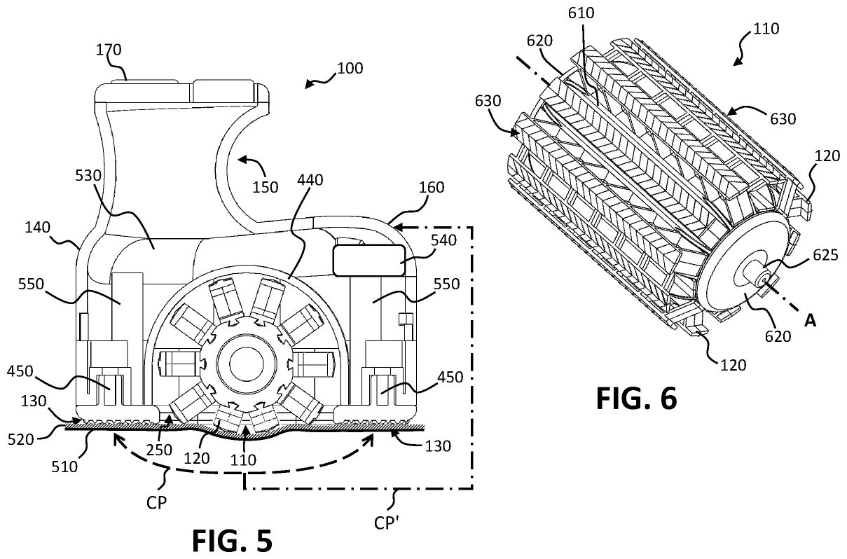

FIG. 5 is a side section view of the skin treatment device 100, showing the roller 110 and compressive contact elements 120, two of which are shown in contact with a subject's skin (or skin surface) 510 (and compressed radially inward, as explained further below). A treatment fluid, conducting gel, or other topical agent 520 can also be applied, and distributed between the bottom surface 130 of device 100 and the top layer of the skin 510.

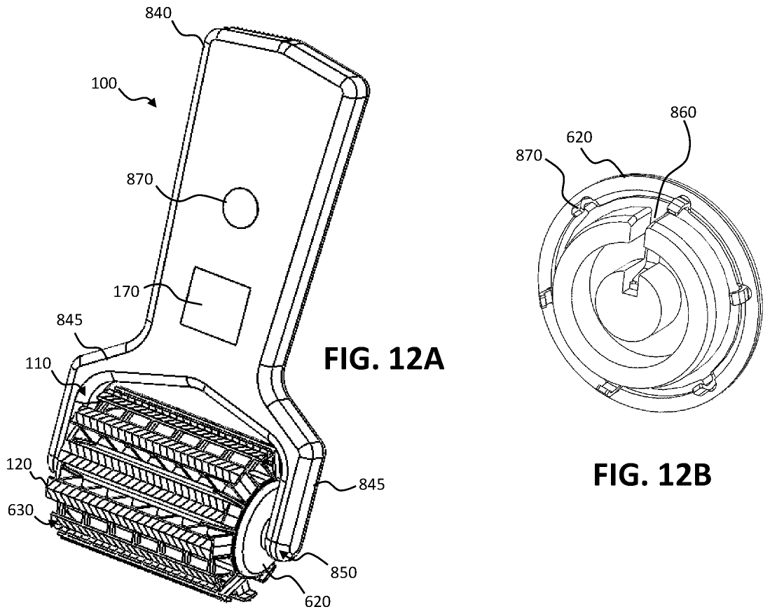

FIG. 5 also shows the other internal components disposed within the device housing 140. In this particular configuration, for example, skin treatment device 100 includes a power supply 530, for example a battery assembly, capacitor bank, or similar power storage system (see also FIG. 12), which is configured to provide power to device 100, and a circuit board or control module 540. The control module (or controller) 540 includes a computer processor or microprocessor, memory and electronic components adapted for operation of device 100, for example a hardware or software driver for user interface 170, a voltage or current supply with control electronics adapted for delivering a microcurrent via a patterned electrode surface 135 (or a plurality of such electrode surfaces 135A, 135B, etc.) and one or more return electrodes 160, and an accelerometer or similar motion sensor(s) adapted to determine the speed of device 100 with respect to the subject's skin, or the rotational speed of roller 110.

Various hard-wired or wireless interface components can also be provided for charging the battery or power supply 530, and for communication of operational and control data with the user or an external device; e.g., tracking device usage via a Bluetooth (or similar short-range) connection to a mobile device running application software specific to the skin treatment device, or via another wired or wireless local network connection to another server or other system used for monitoring or control.

As shown in FIG. 5, Roller 110 is disposed in an opening or aperture 250 in the bottom surface 130 of housing 140; e.g., within a roller housing 440 adapted for rotation of roller 110, isolated from the other internal components of device 100 by a fluid seal. Electrode surfaces 135 (or 135A, 135B, etc.) are formed of a conductive electrode material, and can be connected to the power supply 530 and controller 540 by conducting wires, posts or vias 550. Mechanical fasteners or couplings 450 can be provided to attach bottom surface 130 to the main body of housing 140, for example screws, pins, rivets, or other mechanical attachments. Alternatively, the top and bottom surface 130 of housing 140 can be attached by welding, or with an adhesive.

As device 100 moves across the user's skin, contact elements 120 successively contact the skin surface and compress radially toward the roller axis, providing a beneficial, elastic skin stretching treatment as described herein. In embodiments with two conducting electrode surfaces 135A, 135B, the electrode surfaces can be energized with opposite polarity so that the current path CP passes through the dermal tissue below the subject's skin surface 510, adjacent the electrode surfaces 135A, 135B. The current path CP can thus cross the skin tissue being stretched by the contact elements 120 of roller 110, returning through the opposite electrode surface 135B, 135A. The conducting surfaces of each electrode 135A and 135B can also be energized with the same polarity, or a single electrode surface can be provided (see, e.g., FIG. 3B). In some of these examples, the current path CP′ may extend through the subject's body; e.g., to one or more return electrodes 160 on the upper (or top) side of the device housing 140, spaced from the bottom surface 130, as shown in FIG. 5.

FIG. 6 is an isometric view of a rotor or roller 110 for a skin treatment device 100, showing the individual, flexible, compressive skin contact elements or “skids” 120. Contact elements 120 are distributed circumferentially about roller 110, extending radially about a rotor axle or roller hub 610, and longitudinally along the rotational axis A, between the endcaps 620.

In the representative example of FIG. 6, ten contact elements 120 are distributed uniformly about roller hub 610, extending radially outward from and circumferentially about axis A. There may be more contact elements 120, for example twelve, fourteen, sixteen or more contact elements 120, or fewer contact elements 120, for example four, six or eight contact elements 120. The number of contact elements 120 may also be either even or odd, depending on application (e.g., one, three, five or more contact elements 120), and roller 110 can be replaced with a suitable actuator for application of contact elements 120 to the subject's skin (e.g., a linear or rotary actuator, providing a reciprocating motion, or a combination of rotary and reciprocating motions).

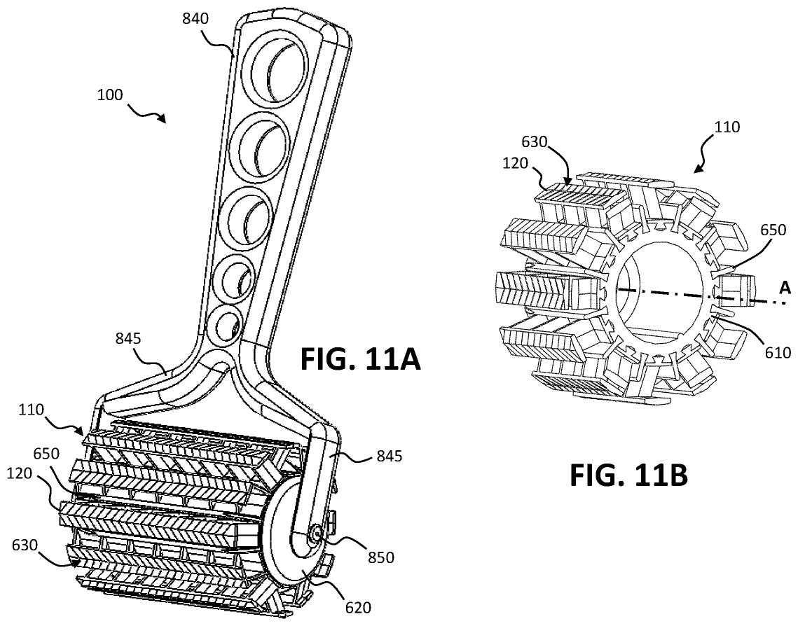

Each roller endcap 620 can be provided with an axial projection or axle (dowel) pin 625 configured to support roller 110 in rotation within the roller housing (see, e.g., FIG. 5), or in an ergonomically designed handle (FIGS. 11A, 12A). Contact elements 120 are provided with a chevron pattern on the outer skin contact surface 630, which is adapted for elastic skin stretching and topical agent distribution as described below. Alternatively, a ridged, scalloped, undulating or wavy surface pattern can be used, or a symmetric grid, radial or spiral pattern, or an asymmetric, irregular, or randomized pattern of round, oblong, triangular, star-shaped, polygonal, or other regular or irregular geometric features can be employed. The pattern on contact surface 630 can also be adapted for cleansing, exfoliating and smoothing the outer skin layers.

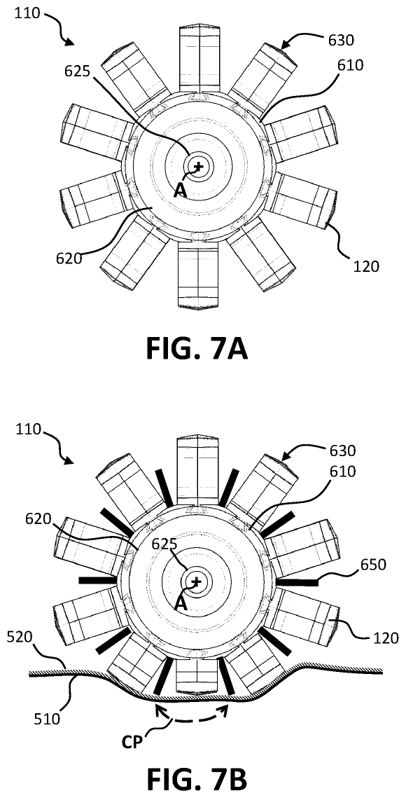

FIG. 7A is a side view of the roller 110, showing the flexible, compressive skin contact elements or “skids” 120 arranged about the rotational axis A, in order to provide a continuous or sequential series of outer contact surfaces 630 when rolled across the user's (or other subject's) skin. FIG. 7B is an alternate side section view of the rotor or roller 110, in an embodiment with bar-shaped electrodes 650 disposed between adjacent contact elements 120. As shown in FIG. 7B, the electrodes or bars 650 are distributed circumferentially about the body of roller 110, with conducting surfaces extending longitudinally along the roller axis A, and interleaved or interspersed between pairs of adjacent contact elements 120. In these examples, the bars 650 can be electrified, so that roller 110 provides a current-based skin treatment. The bars 650 also serve a mechanical function, acting to set the maximum compression distance of the contact surfaces 630 in the radial direction (that is, toward the rational axis A), based on the radial height of the adjacent bars 630. The bars 630 can also help prevent the elements 120 from flexing or bending in the circumferential direction (as defined about the axis A), maintaining the radial orientation of the compression, and the axial orientation of the lateral displacement.

As roller 110 moves over a subject's skin (or other surface to be treated) 510, contact elements 120 successively compress radially toward axis A, bringing the conducting surfaces of the bars 650 into physical and electrical contact with the skin surface 510, either directly or indirectly (e.g., through a conducting gel, fluid, or other topical agent 520 distributed between contact surfaces 630 on contact elements 120, and the outer layers of the subject's skin 510). In operation, two bars 650 can be brought into simultaneous physical and electrical contact with the skin surface 510 when the adjacent contact elements 120 are compressed, forming a current path through the topical agent 520 and adjacent skin surface 510. When an electrical treatment is not desired, bars 650 can still act to mechanically constrain the radial compression of contact surfaces 630, and to limit displacement of the flexible elements 120 to the radial (compressive) and lateral (axial) directions, reducing or preventing circumferential bending and distortion.

Alternating pairs of the bar electrodes 650 can be energized with opposite polarity, and adapted to provide a beneficial current or microcurrent treatment to the subject's skin 510 as described above, for example by providing a battery and circuit board with suitable power and control electronics within the body of the roller 110. In these examples, adjacent pairs of the bars 650 can be controlled to have opposite polarity, and the voltage or current waveform can be adapted for the smaller contact area and shorter current path CP; e.g., passing substantially or mainly through the dermal tissues below skin surface 510, between the adjacent bars 650.

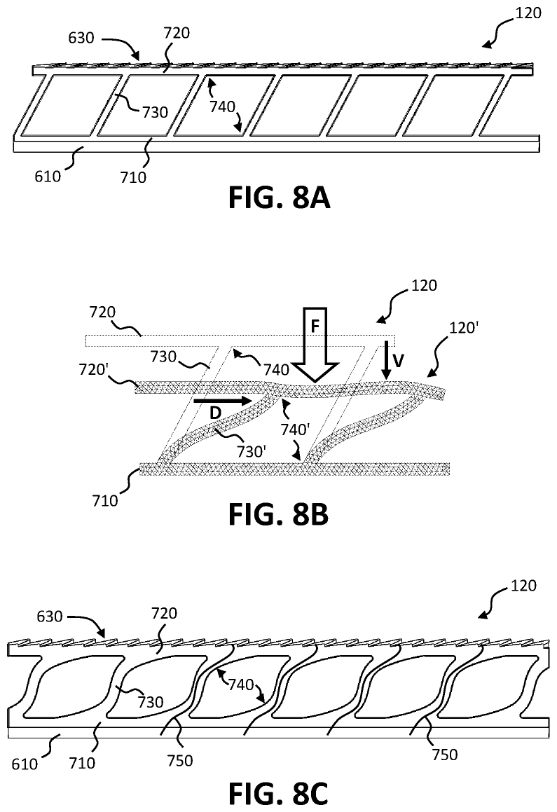

FIG. 8A is a side elevation view of a compressive contact element 120 for a roller 110, as described herein. As shown in FIG. 8A, contact element 120 includes a first longitudinal portion or lower (radially inner) flange section 710 coupled to the roller hub 610, and a second longitudinal portion or upper (radially outer) flange section 720, coupled together with a series of transverse web sections or web elements 730.

A skin contact surface 630 is formed on the outer (top) surface of the upper (second) flange section 720, opposite the lower (first) flange section 710 and roller hub 610. The web elements 730 extend at an angle between the upper and lower flange sections 710, 720, for example at a skew angle defined at or adjacent the couplings 740 between the web elements 730 and flanges 710, 720. As seen in FIGS. 8A and 8C, the transverse web portions connecting between the first longitudinal portion or lower (radially inner) flange section 710 coupled to the roller hub 610, and the second longitudinal portion or upper (radially outer) flange section 720 form a generally ladder-like configuration.

The angle of web element or web member 730 at the couplings 740 can be selected so that top flange section 720 displaces both vertically (radially) and horizontally (axially or longitudinally) in response to a compressive load applied at or across the opposed surfaces of the longitudinal flange sections 710, 720. As a result, the contact surface 630 also displaces, in a direction from left to right as shown in FIG. 8A. Suitable skew angles depend upon the desired amount of displacement that required or selected in a particular application, for example about 110° to about 120°, about 100° to about 130°, or up to 150° or more.

Contact elements 120 can be formed of a resilient, flexible, elastic polymer or composite material, selected to withstand repeated cycles of bending, compression and expansion without loss of strength, and able to repeatedly and reliably return to their original shape when the compressive force is removed. The orientation of adjacent contact elements 120 can be alternated about the roller, so that the adjacent contact surfaces 630 are displaced in opposite directions, along with the adjacent skin in contact with the surfaces 630, providing a beneficial, elastic stretching effect to the skin along and between the outer surfaces 630 of the adjacent compressive skin contact elements 120.

FIG. 8B is a side elevation view illustrating compression of a representative flexible, compressive skin contact element 120 upon application of a perpendicular load force F, for example in response to contact with the subject's skin. As shown in FIG. 8B, contact element 120 is compressed in response to the load F, forming a new configuration 120′ with upper flange section 720 displaced vertically downward (or radially inward), and horizontally (longitudinally, along the rotor axis), as represented by the compressed configuration of the upper flange section to new configuration 720′ with lateral displacement D to the right, and vertical, downward displacement V.

As seen in FIG. 8B, the web elements 730 bend downward toward a new compressed configuration 730′, increasing the skew angle adjacent the coupling 740 to a new compressed configuration 740′. The lateral (or longitudinal) displacement D of the upper flange portion 720 increases with vertical (or radial) displacement V, up to a limit defined by the compressive force F, and the length, thickness, number, and flexibility of the web elements 730.

This increases stress on the corner couplings, which can induce fatigue in the material properties over repeated compression cycles. To increase strength and service life, the cross section of the web elements 730 can be relatively greater at or adjacent the couplings 740, and relatively less in the medial portion extending between the couplings 740. Similarly, the flexibility of the web element 730 may be greater in the medial portion than at or adjacent the couplings 740.

FIG. 8C is a side elevation view of an alternate compressive contact element 120 for a roller 110, as described herein. In this example, web elements 730 have a non-linear “S” shape with increased skew angle and material thickness (cross section) at or adjacent couplings 740, where the web elements 730 connect to the upper and lower longitudinal portions of compressive element 120, formed by the first and second flange sections 710 and 720. One purpose of this configuration is to increase or maximize the range of lateral movement (horizontal displacement D) with a lesser or least amount of radial compression (vertical displacement V). These geometric modifications were based on finite element analysis (FEA), using the elasticity and other physical properties of the materials selected for forming the flange portions 710, 720, web members 730 and couplings 740 of the compressive elements 120, and based on the geometry shown in FIG. 8C. Other configurations are also contemplated, as adapted to maintain, increase or maximize the range of transverse motion D, as compared to vertical (radial) compression V (e.g., as defined by an increased ratio D/V). This configuration can also increase resistance to repeated stress cycles, while having reduced cross section in the medial portion extending between the couplings 740, providing material flexibility to maintain or increase the horizontal displacement response.

A suitable voltage or current signal can also be provided to electrodes on the outer surfaces 630 of the compressive skin contact elements 120; e.g., via flexible conductors 750 connecting to the outer contact surfaces 630; e.g., via the radially inner and outer longitudinal portions (flanges) 710, 720 and transverse web elements 730 of the respective contact elements 120. In these examples, the current signal can be provided via the rotor hub 610, adjacent the rotor axle, from which a suitable voltage or current signal may be delivered to the electrodes via conductors 750. Additional conductor or conducting elements 750 can also be disposed along the rotor hub 610, and/or along the inner and outer longitudinal portions (flanges) 710, 720 of the contact elements 120, in order to distribute the current flow.

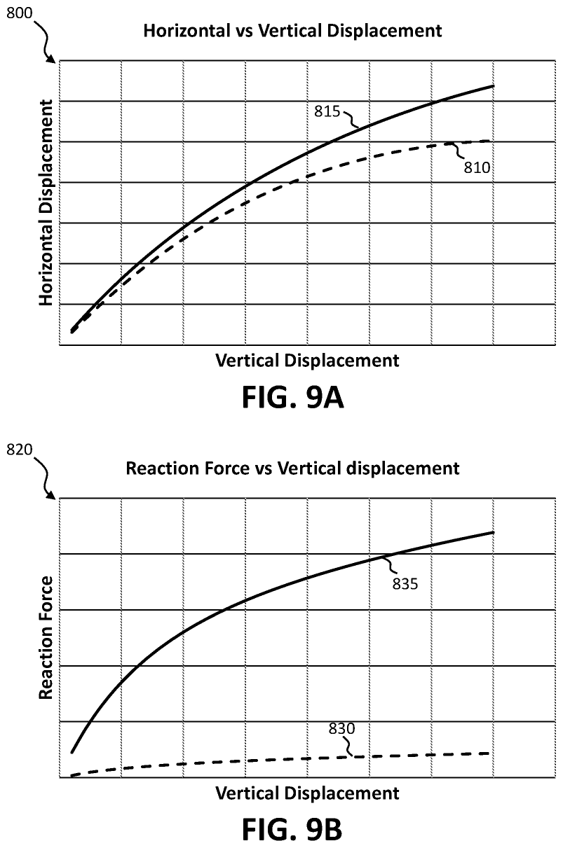

FIG. 9A is a plot (800) of horizontal travel distance or displacement versus vertical displacement for representative skin contact elements represented by dashed line 810 and solid line 815; e.g., according to FIGS. 8A and 8B, respectively. Travel distance (lateral or horizontal displacement) is shown on the vertical axis and vertical displacement (radial compression) on the horizontal axis, both in arbitrary units.

FIG. 9B is a plot 820 of reaction force versus vertical displacement for the representative skin contact elements represented by dashed line 830 and solid line 835; e.g., according to FIGS. 8A and 8B, respectively. Reaction force is shown on the vertical axis and displacement on the horizontal axis, both in arbitrary units. The reaction force can be defined as the magnitude of the normal applied by the user's skin onto the contact surface of the respective skin contact element, resulting in a radially compressive load on the skin contact elements, causing them to compress radially toward the roller axis, or equivalently as the force applied by the contract surfaces onto the subject's skin.

As shown in FIGS. 9A and 9B, compression of the skin contact elements causes horizontal displacement of the respective skin contact surfaces, in response to compressive load or vertical displacement. The relationship between vertical displacement (radial compression toward the roller axis) and horizontal displacement (along the skin surface) can be selected based on geometry of the contact element, including the geometry of the transverse web sections, and the corner couplings where the web sections connect to the upper (radially outer) and lower (radially inner) flanges. The load response or “feel” of the compression can also be adapted based on geometry of the skin contact element, and by selecting materials with different young's modulus and other elastic properties. This allows the skin treatment system to be adapted for roller size and user preference (e.g. “firmer” or “softer” action, based on the longitudinal or transverse displacement response to a given (higher or lower) compressive (radial) loading or reaction force.

The typical horizontal and vertical displacements vary depending on roller size and application, In some examples, the displacements range from about 0.5 mm or less to about 3.5 mm or more, for example from about 0-3.5 mm, from about 1.0-5.0 mm, or up to 5.0-10 mm or more. The reaction force also varies, for example from about 0.5 newton (N) or less to about 2.5 N or more, for example from about 0-2.5 N, from about 1.0-3.0 N, or up to 3.0-5.0 N or more.

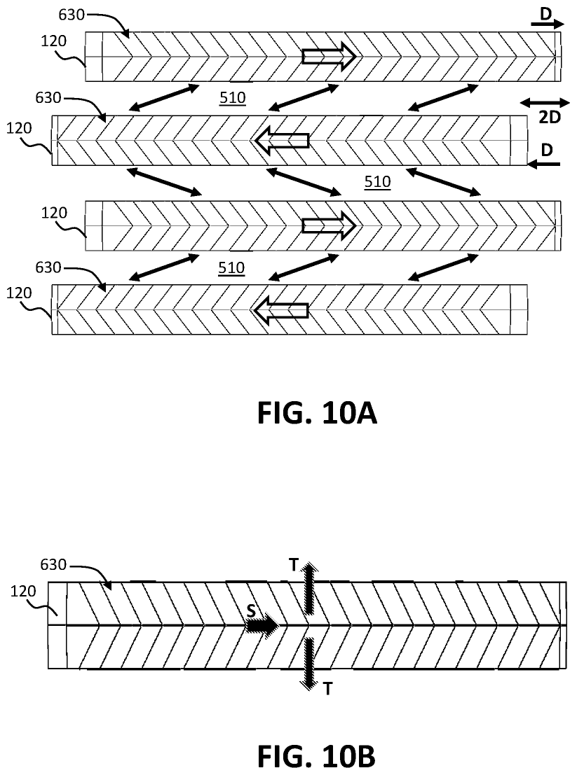

FIG. 10A is a schematic diagram illustrating the origin of the skin stretching effect of the contact elements 120, in operation of a roller mechanism 110 as described herein. As shown in FIG. 10A, adjacent pairs of contact elements 120 can be arranged with reversed orientation, causing the respective contact surfaces 630 to displace in different directions in response to compressive contact with the subject's skin, or other treatment surface. This displaces the adjacent portions of the skin surface 510, imposing a tensile load and causing the skin 510 to stretch between the adjacent pairs of contact elements 120.

Typically, at least two (and possibly more) adjacent compressive elements 120 can contact the skin at any given time, as the roller is manipulated over the treatment surface. As successive compressive elements 120 make contact, the skin is alternately stretched and then released, providing a beneficial treatment designed to improve skin tone and appearance, while reducing undesirable effects. Chevron or ridged patterns can also be provided on contact surfaces 630, and adapted to improve distribution of the topical agent over the skin surface, modulate the friction between contact surfaces 630 and the skin 510, clean the surface of the skin 510, and increasing absorption of skin moisturizers, nutrients and other beneficial agents in the topical fluid.

The horizontal or lateral displacement D of individual skin contact surfaces 630 along the skin surface 510 is merely representative, and increases with compression and vertical displacement of the respective contact elements 120. The total relative displacement resulting from compression (vertical displacement) of the skin and horizontal (or lateral) displacement occurring between two adjacent contact elements 120 is twice the displacement distance D of an individual contact surface 630 (see also, e.g., FIG. 8B). If all of the relative displacement in each of the two adjacent contact surfaces 630 were transferred to the skin, the total relative displacement of adjacent skin surfaces 510 (disposed between adjacent, displaced contact surfaces 630) would be twice the displacement distance D.

The skin displacement (or stretching) also depends, however, on the viscosity and lubrication properties of any topical agents or fluids applied to the skin, and the compressive load (or normal force) on the skin contact surfaces 630 and adjacent skin surface 510, as well as on changes in the skin elasticity responsive to the applied stretching effect. The movement of the skin surface 510 contacted by the respective contact surfaces 630 of adjacent pairs of contact elements 120 may thus transition from an initial phase of static friction, where the skin surface 510 maintains contact with the adjacent outer surfaces 630, to a kinetic friction phase, where the contact surfaces 630 slip or slide across the adjacent skin surfaces 510.

The total relative displacement of the two adjacent, displaced skin contact surfaces 630 may thus not be fully effected in or on the skin surface 510 between the adjacent contact surfaces 630. This allows the user to increase, decrease, or otherwise control or select the desired amount of skin stretching; e.g., by increasing or decreasing the compressive load or pressure (normal force) on the skin surface 510, or by using a selected lubricating agent or other topical on the skin surface 510, in order to maintain a comfortable, beneficial degree of skin stretching in skin surface 510, and to avoid undesirable irritation.

FIG. 10B is a schematic diagram illustrating distribution of a topical fluid by a flexible, compressive contact element 120, in operation of a roller mechanism 110 as described herein. As shown in FIG. 10B, the chevron pattern on skin contact surface 630 will typically remain in contact with the skin for a portion of the horizontal displacement, contributing to the elastic stretching treatment, and then the contact surface 630 will begin to slip, once the skin has stretched to a particular degree and some of its elasticity is used.

Depending on application, this can result in a combination of sticking (static friction between skin contact surface 630 and the subject's skin) and slipping along the skin surface (sliding or kinetic friction), which combine to determine the total skin displacement (or stretching effect). When static friction dominates, for example, the skin displacement or stretching effect on the skin can be up to twice the relative (opposite) displacements of the adjacent contact surfaces 630; e.g., up to 2.0-10.0 mm or more. Where there is substantial slipping (kinetic friction), the skin displacement or stretching effect on the skin can be somewhat less, for example 1.0-5.0 mm, or more or less.

The relative contributions of the “sticking” (static friction) phase and the slipping (kinetic friction) phase depend upon the normal (or “reaction”) force applied to compress the contact elements 120 against the subject's skin, as well as the corresponding coefficients of static and kinetic friction between skin contact surface 630 and the skin. The coefficients of friction, in turn, depend upon the interaction of the textured pattern on contact surface 630 with the subject's skin, as well as the density, viscosity, and other fluid properties of any topical treatment that is applied between the skin and contact surface 630. In some examples, the topical treatment and roller parameters can be selected to adjust the relative contribution of the static and kinetic phases, for example by selecting a “firmer” or “softer” roller with relatively greater or lesser normal force required for compression of the contact elements 120, and by selecting topical treatments with different viscosity and other fluid properties.

The amount of stretching depends on the skin properties and the coefficients of static and kinetic friction defined between the skin contact surface 630 and the subject's skin, which in turn depends on the amount and quality of the topical agent, including agent thickness or depth, temperature, viscosity and related lubrication parameters. Once the contact surface 630 begins so slip (or slide, in sliding frictional contact) over the skin surface (arrow A), the pattern will distribute the topical agent in the transverse directions, toward either side of the contact element 120. This action improves distribution of the topical agent, providing greater control over the coefficients of friction and improving uniformity of the degree of skin stretching that is achieved, over the selected treatment area. The slipping of the textured pattern features along the subject's skin surface (e.g., chevron, ribbed, ridged, edged or other features) can also provide a wiping or “squeegee” effect at or along the skin surface, which can help remove dirt, loose skin cells or other debris from the outer surface of the subject skin, as well as materials released from the skin pores.

FIG. 11A is an isometric view of an alternate skin treatment device 100, with roller 110 supported in an ergonomic handle 840. FIG. 11B is an isometric view of a roller 110 for the device of FIG. 11A, with a number of mechanical bar-shaped members 650 distributed circumferentially about the body of the rotor or roller 110, extending longitudinally along the roller axis, interleaved between adjacent pairs of the contact elements 120. Mechanical bars 650 can be configured to limit the radial compression of the contact surfaces 630, and to reduce distortion of compressive elements 120 in the circumferential direction, while maintaining lateral displacement, as described above.

In the example of FIGS. 11A and 11B, handle 840 is adapted for manipulation of device 100 by a user's hand, with roller 110 supported between two forks or prongs 845 extending from the main body of the handle 840. Roller 110 can be rotationally supported by one or more bearing assemblies 850 disposed in the ends of the prongs 845, and adapted to engage an axle, pin, or similar extension on the roller endcaps 620, along the rotational axis A of roller 110 (see also, e.g., FIGS. 8A-8C).

A number of bar-shaped mechanical elements 650 can be disposed about roller 110, for example between adjacent flexible contact elements 120 as shown in FIG. 11B. In operation, one, two or more such bars 650 can make physical contact with the subject's skin at any given time, when the adjacent pairs of contact elements 120 are compressed down toward or to the height of the adjacent bar or bars 650. A topical agent can also be applied to the subject's skin, as described above.

FIG. 12A is an isometric view of a skin treatment device 100 with a roller 110 supported in an active handle 840, and adapted to provide a combination of elastomeric stretching and current-based skin treatment. FIG. 12B is a detail view of a roller endcap 620 for device 100 of FIG. 12A.

In these examples, a number of bar-shaped electrodes 650 are provided on roller 110, disposed between adjacent pairs of the contact elements 120. A battery or other power source and control circuitry can be provided in a “smart” or active handle 840; e.g., with a user interface 170 and additional control elements such as an on/off control 870. Roller endcaps 620 can also include a location feature or key 860 to orient the endcap to the roller, and an electrical ring contact 870 or similar contact structure providing electrical contact with the bar-shaped electrodes 650.

For example, ring contacts 870 can be provided with different keys 860 on opposing roller endcaps 620, and adapted to deliver opposite polarity current to selected subsets of the alternating electrodes 650. The opposite endcaps 620 thus have a complementary arrangement, in contact with alternating (oppositely polarized) electrodes 650, for example using a metal axle or pin 625, or similar conducting, rotational bearing element 625, configured to support roller 110 in rotation within a bearing assembly in handle 840, and for electrical connection to a power supply and control electronics in handle 840.

In other embodiments the electrodes may take the form of electrical contacts disposed in or on all or a portion of the skin contact surfaces 630 on one or more adjacent pairs of contact elements 120, or on each or all of the contact elements 120. In these examples, a suitable current path can include areas of the subject's skin (or other surface) disposed between two adjacent contact elements 120, in frictional engagement with the adjacent skin surface along the outer contact surfaces 630.

For example, current may be provided by flexible conductors on the outer surfaces 630 of the contact elements 120, or embedded in the transverse web members connecting to the radially outer longitudinal portions of the respective contact elements 120, adjacent the rotor axle, from which a suitable voltage or current signal may be delivered to the electrodes 650. Conducting elements can also be disposed in or on the inner and outer longitudinal portions of the contact elements 120, disposed radially inward and outward from the roller axis, respectively, which form the substrate for a radially inner connection to the rotor bub, and for the outer contact surfaces 630 that deliver current to adjacent skin areas (see, e.g., FIGS. 8A-8C).

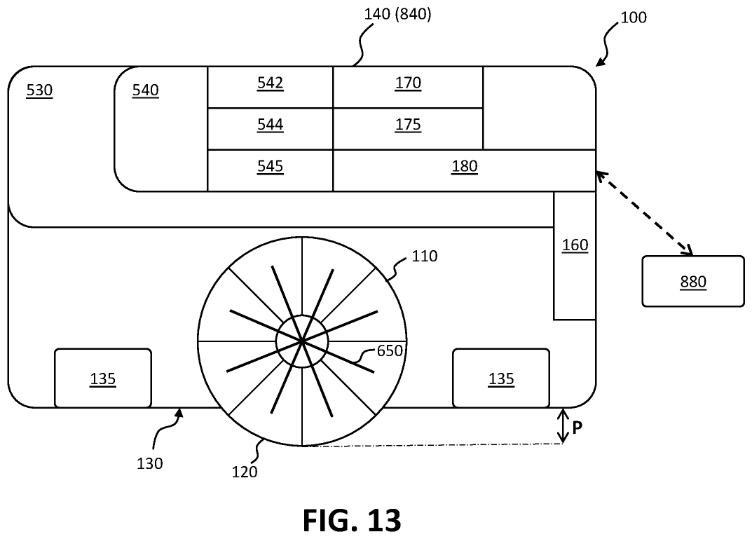

FIG. 13 is a schematic block diagram of a representative skin treatment device (or skin treatment system) 100, as described herein. As shown in FIG. 13, device 100 includes a roller 110 at least partially disposed within a housing 140; e.g., with a plurality of compressive contact elements 120 disposed about a rotational axis. Roller 110 can also be rotationally supported on a handle 840, which can be configured to provide one or more functions of the housing 140.

One or more electrodes can be provided, for example one or more treatment electrodes (or electrode surfaces) 135 on the bottom surface 130 of device 100, one or more bar-shaped electrodes 650 on roller 110, and one or more return electrodes 160 on housing 140. A battery or other power supply 530 and controller 540 can also be provided, for example a rechargeable battery system or capacitor bank 530 with a controller having a computer processor or microprocessor 542, memory 544 and a voltage or current supply 545 adapted for delivering a current or microcurrent treatment via one or more of the electrode surfaces 135, 160 and 650, as described herein.

The schematic diagram of FIG. 13 also indicates that roller 110 protrudes or extends outwardly a certain distance P from the plane of the bottom surface 130 of the housing 140, where the treatment electrodes or electrode surfaces 135 can be located. This outward extension distance P may in some embodiments be made adjustable by mounting the axle of the roller 110 in any suitable arrangement of paired support hubs for the axle ends, which can be adjusted along a path or track that is oriented in a vertical direction (perpendicular to the bottom surface 130 of the housing 140), or which has a vertical component that is transverse to the bottom surface 130 (e.g., at a skew angle).

Such an adjustable mounting for roller 110 would facilitate adjustment of the outward extension distance P. This adjustment would allow increasing or decreasing the maximum amount of compression displacement that could be applied to the contact elements 120, which interact with the subject's skin as shown (e.g., as shown in in FIGS. 5 and 6C). This roller height adjustment could be used to select the extension distance P for user comfort, or to accommodate rollers 110 with contact elements 120 and electrodes or mechanical bars 650 of different dimensions, or with different resilience and response to a compressive load, or other structural features and material qualities.

Controller 540 can also include additional electronic components adapted for operation of device 100, including a driver 175 for a user interface 170 with a graphical display, and/or a wired or wireless interface 180 adapted for operational and control communication with a smart phone, tablet, mobile computing device, or similar user device, or a server 880 configured for communication with device 100 via interface 180. The user device or server 880 can be configured to execute application software specific to control, monitoring, display and other operations of the skin treatment device 100, as described herein.

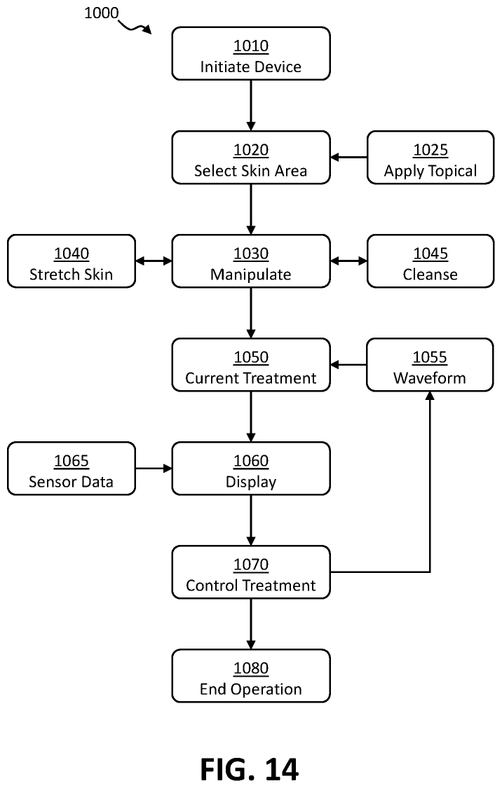

FIG. 14 is a block diagram illustrating a method 1000 for operating a skin treatment device or system 100, as described herein. As shown in FIG. 14, method 1000 includes one or more steps or processes of initiating operation of the device (step 1010), selecting a skin area for treatment (step 1020), applying a topical agent (step 1025), and moving or manipulating the device across the skin surface (step 1030), in order to provide a beneficial elastic skin stretching treatment (step 1040), to cleanse or exfoliate the skin (step 1045), or both.

In some examples, method 1000 also includes one or more steps of selecting and/or applying a current treatment (step 1050), for example using a voltage or current waveform (step 1055), and communicating operational parameters for display (step 1060); e.g., on an internal user interface or external user device (step 1060). In practice, the current treatment 1050 can be provided concurrently with stretching (step 1040) and cleansing (step 1045); e.g., as the device is manipulated over the subject's skin (step 1030). Sensor data can also be collected and displayed (step 1065), and the operational parameters can be controlled or adjusted (step 1070), before ending operation of the device (step 1080). The steps of method 1000 can be performed in any order or combination, with or without additional skin treatment processes, steps and techniques, as described herein.

Depending on application, for example, method 1000 can be performed by initiating the device (step 1010) via a user interface on the device, or responsive to instructions received from an external user device such as a smart phone, tablet computer, or other personal computing device, in wired or wireless communication with a hardware interface on the skin treatment device 100.

A skin area is selected for treatment (step 1020), either by the subject, or by another user applying the device to the subject's skin. In many applications, a topical agent is applied to the selected skin area (step 1025), for example a topical fluid with one or more skin moisturizers, nutrients, collagen, and other beneficial skin treatment components. Alternatively, a topical agent may not be required.

The device can be moved or manipulated (step 1030) via a handle configured to rotationally support the roller, for example at a hub or axle, or via a suitable housing with a bottom surface through which a portion of the roller extends. As the roller moves across the skin surface, a load is sequentially applied to the flexible compressive skin contact elements. In response to the load, the elements displace radially toward the rotational axis of the roller, and laterally along the skin surface.

The lateral displacements of adjacent pairs of the flexible compressive elements are defined in opposing directions, transverse to the direction of the load, and parallel or antiparallel to the rotational axis, respectively. This provides an elastic stretching treatment (step 1040) to a portion of the skin surface between the adjacent flexible compressive elements, by generating a tensile load on the skin surface, responsive to the compressive load on the flexible skin contact elements. The lateral displacement of the skin contact elements can also provide a cleansing or exfoliating treatment to a portion of the skin surface adjacent the skin contact elements (step 1045). In some applications, this step can include distributing the topical agent or other skin treatment along the skin surface, and encouraging absorption into the skin.

The skin treatment device can also configured to apply a current treatment to the skin surface (step 1050); e.g., a microcurrent or galvanic treatment, via a number of conducting surfaces or electrodes. For example, one or more conducting surfaces or electrodes can be disposed on a bottom surface of the device housing, through which the flexible compressive elements sequentially extend as the device is manipulated across the subject's skin. Additional conducting surfaces or electrodes can also be disposed on the bottom surface, for example on opposing sides of the roller, or on the handle or upper surface of the housing, spaced from the bottom surface, with the same or opposite polarity. Conducting surfaces or electrodes can also be disposed between the adjacent flexible compressive elements, or on the radially outer skin contact surfaces.

A voltage or current waveform can be generated (step 1055) for application of the current treatment, for example using operational parameters communicated for display an internal user interface or external user device (step 1060). Suitable operational parameters can include, but are not limited to, a time duration or remaining time of the current treatment, a voltage or current level of the current treatment, a pulse width of the current treatment, and a record of a prior instance of the current treatment as applied to the skin surface, for example by date and time, or an accumulated treatment time over a selected period (e.g., a day, week, month, quarter, or year).

Sensor data can also be generated and displayed, either on the user interface or an external user device (step 1065). Suitable sensors can collect data related to conditions of the subject's skin, for example resistivity or elasticity of the skin surface, force or pressure on the skin surface (e.g., responsive to the compressive load), temperature at or proximate the skin surface, and/or ambient temperature or humidity at or proximate the skin surface.

The waveform and current treatment can be initiated, terminated, or controlled (step 1070); e.g., via an internal user interface, or with an external user device such as a smart phone, tablet computer, or other mobile or personal computing device. For example, the user can adjust a time duration, voltage level or current level of the current treatment, or control a pulse width, period, frequency, or duty cycle of the waveform.

Once the treatment is complete, operations can be ended (step 1080). Before shutting down, the device can store operational parameters related to the skin treatment in memory, for example date, time and duration of the skin cleansing and current treatments, and any or all of the sensor data. The stored operational conditions can also include voltage and current levels related to the current treatment, as well as pulse width, period, frequency, and other waveform parameters, as described above. These data can be stored in memory included with a user interface or controller on the device, or communicated to an external user device, for display on either platform.

The examples and embodiments of the invention described here can be practices either alone or in combination with any of the other examples and embodiment that are described, and may incorporate other modifications, equivalents, and alternatives falling within the language of the claims. The various disclosed embodiments are provided by way of illustration, and should not be construed to limit the scope invention except where plainly recited in the claims. Various modifications and changes can be made to the embodiments and applications illustrated and described herein, without departing from practice of the inventions as claimed.

1. A device comprising:
a roller disposed about a rotational axis;
a plurality of compressive elements distributed circumferentially about the roller and extending radially therefrom, each of the compressive elements comprising:
a first, radially inner longitudinal section coupled to or adjacent the roller;
a second, radially outer longitudinal section having a skin contact surface opposite the first longitudinal section, and
a transverse section comprising one or more web elements extending between the first and second longitudinal sections;
wherein the web elements are adapted for radial and lateral displacement of the second longitudinal sections in response to a compressive or radial load, such that the contact surfaces of one or more adjacent pairs of the compressive elements displace in opposing directions along or parallel to the rotational axis, and transverse to a direction of the load.

2. The device of claim 1, wherein one or more of the web elements are skewed at an angle with respect to the first and second longitudinal members, the angle having different orientations in one or more of the adjacent pairs of the compressive elements to define the opposing directions along or parallel to the rotational axis.

3. The device of claim 1, wherein the web elements are skewed an angle at or adjacent couplings to the first and second longitudinal members, each of the web elements having a medial portion extending between the couplings, wherein:
a cross section of the web element is greater at or adjacent the couplings than in the medial portion; and/or
a flexibility of the web element is greater in the medial portion than at or adjacent the couplings.

4. The device of claim 1, wherein the contact surfaces are textured for frictional contact with a skin surface under treatment by which the load is applied, and:
wherein the textured contact surfaces comprise a ridged, scalloped, undulating, symmetric grid, spiral, radial, asymmetric, irregular, or randomized pattern extending across the one or more radially outer surfaces, adapted to provide the frictional coupling;
wherein the textured contact surfaces are adapted to stretch a portion of the skin surface between one or more of the adjacent pairs of the compressive elements, in response to the load; and/or
wherein the textured contact surfaces are adapted to cleanse, exfoliate, or distribute a topical agent over the skin surface, responsive to the lateral displacement.

5. The device of claim 1, further comprising a housing defined about the roller and having an aperture through which one or more of the compressive elements extend, wherein the housing is configured for manipulation of the device over a skin surface adjacent the device.

6. The device of claim 5, further comprising one or more conducting surfaces or electrodes configured to define a current path through the skin surface adjacent the device, wherein the one or more conducting surfaces or electrodes comprise:
at least one conducting surface or electrode having a patterned treatment surface extending along a bottom surface of the housing, adjacent the roller;
first and second conducting surfaces or electrodes having opposite polarity, and configured for electrical contact with the skin surface upon application of the load;
first and second conducting surfaces or electrodes disposed along a bottom surface of the housing, on opposite sides of the roller; and/or
a first conducting surface or electrode configured for electrical contact with the skin surface on a bottom of the housing adjacent the roller, and a second conducting surface or electrode configured for electrical contact with a hand or other body portion of the subject on a handle or upper surface of the housing, spaced from the bottom surface.

7. The device of claim 6, further comprising one or more of a controller, a power source, a voltage generator, or a current generator disposed in or on the housing, and configured to generate a voltage or current waveform for application of a current treatment to the skin surface via one or more of the conducting surfaces or electrodes.

8. The device of claim 1, further comprising a housing or handle configured to support the roller, wherein the housing or handle is configured to generate the load by manipulation of the device over a skin surface of a subject.

9. The device of claim 8, further comprising one or more conducting surfaces, bars or electrodes configured for electrical contact with the skin surface, wherein:
one or more of the conducting surfaces, bars or electrodes are disposed between the adjacent compressive elements, and configured for electrical contact with the skin surface upon application of the load;
one or more of the conducting surfaces, bars or electrodes are disposed in or on one or more of the radially outer surfaces of the compressive elements; and/or
one or more of the conducting surfaces, bars or electrodes are disposed in or on the housing or the handle.

10. The device of claim 9, wherein the one or more conducting surfaces, bars or electrodes are configured for application of a current treatment to the skin surface, and further comprising:
one or more of a power source, a voltage generator, a current generator or a controller disposed in or on the housing, the handle or the rotor, and configured to generate a voltage or current waveform for application of the current treatment to the skin surface via the one or more conducting surfaces, bars or electrodes;
a user interface or display disposed in or on the housing, the handle or the rotor, and configured to control or display one or more of a period, frequency, voltage amplitude, current amplitude, or time duration of the current treatment; and/or
a user device interface disposed in or on the housing, the handle or the rotor, and configured to communicate one or more of a period, frequency, voltage amplitude, current amplitude, or time duration of the current treatment with an external user device, or to control the application of the current treatment via the external user device.

11. The device of claim 8, further comprising one or more sensors disposed in or on the housing, the handle or the rotor, the one or more sensors selected from:
a motion sensor or accelerometer configured for sensing motion of the device over the skin surface;
a rotational sensor or encoder configured to sense a rotational speed or rotational position of the rotor;
a temperature sensor configured for measuring temperature at or proximate the skin surface;
a humidity sensor configured for sensing humidity at the skin surface or proximate the device;
a moisture sensor configured for sensing a moisture level associated with the skin surface;
a conductivity sensor configured for sensing conductivity of, at or through the skin surface; and/or
an elasticity sensor configured for measuring elasticity of the skin surface.

12. A method of treating skin, comprising:
manipulating a roller across a skin surface of a subject, the roller having a plurality of flexible compressive elements distributed circumferentially about a rotational axis and extending radially therefrom; and
applying a load to one or more of the flexible compressive elements via the skin surface, wherein the flexible compressive elements are configured for radial displacement toward the rotational axis and lateral displacement along the skin surface, in response to the load;
wherein the lateral displacements of one or more adjacent pairs of the flexible compressive elements are defined in opposing directions transverse to a direction of the load, along or parallel to the rotational axis.

13. The method of claim 12, further comprising:
providing an elastic stretching treatment to a portion of the skin surface between one or more of the adjacent pairs of the flexible compressive elements, responsive to the load;
cleansing, exfoliating, or distributing a topical agent over the skin surface, responsive to the lateral displacement thereof; and/or
cleaning or exfoliating a portion of the skin surface, responsive to the lateral displacement thereof.

14. The method of claim 12, further comprising applying a current treatment to the skin surface via one or more conducting surfaces or electrodes, wherein:
one or more of the conducting surfaces or electrodes comprise a patterned treatment surface extending over at least a portion of a bottom surface of a device housing, adjacent the roller;
one or more of the conducting surfaces or electrodes is disposed on a bottom surface of a housing through which one or more of the flexible compressive elements extend, and one or more other of the conducting surfaces or electrodes is disposed on a handle or upper surface of the housing, spaced from the bottom surface;
one or more of the conducting surfaces or electrodes are disposed between the adjacent flexible compressive elements; or
one or more of the conducting surfaces or electrodes are disposed in or on a radially outer surface of one or more of the flexible compressive elements.

15. The method of claim 14, further comprising generating a voltage or current waveform for application of the current treatment via the one or more conducting surfaces or electrodes, and:
displaying operational parameters related to the current treatment with a user interface or user device, the operational parameters selected from one or more of: a time duration or remaining time of the current treatment, a voltage or current level of the current treatment, a pulse width, frequency, or duty cycle of the voltage or current waveform, or a record of prior instances of the current treatment, as applied to such a skin surface;
displaying sensor data related to the skin surface on a user interface or user device, the sensor data selected from one or more of motion of the device across the skin surface, a rotational position of the roller with respect to the skin surface, resistivity or elasticity of the skin surface, force or pressure on the skin surface, responsive to the compressive load, temperature at or proximate the skin surface, and ambient temperature or humidity at or proximate the skin surface; and/or
initiating or terminating the current treatment, or controlling or adjusting a time duration, voltage level or current level of the current treatment, or a pulse, period, frequency, or duty cycle of the voltage or current waveform, responsive to user input from a user interface or user device.

16. A skin treatment device comprising:
an axle or hub disposed about or along a rotational axis;
a plurality of skin contact elements extending radially from the axle or hub, each of the skin contact elements comprising:
a first longitudinal portion coupled to or adjacent the axle or hub;
a second longitudinal portion having a radially outer skin contact surface disposed in spaced, parallel relationship to the first longitudinal portion; and
one or more flexible web elements extending transversely between the first and second longitudinal portions, at an angle thereto;
wherein the angles of the web elements are adapted for the second longitudinal portions of the skin contact elements to displace radially inward and laterally along the rotational axis relative to the first longitudinal portions, in response to a radial or compressive force on the respective contact surfaces.

17. The skin treatment device of claim 16, wherein:
the angles are skewed with an opposing orientation in one or more adjacent pairs of the skin contact elements, such that the respective second longitudinal portions displace laterally in opposing directions, along or parallel to the rotational axis, and transverse to the force; and/or
the radially outer contact surfaces are adapted for frictional contact with an adjacent skin surface by which the force is applied, and for generating a tensile load to stretch a portion of the skin surface between the one or more respective adjacent pairs of the skin contract elements, responsive to the frictional contact.

18. The skin treatment device of claim 16, further comprising a surface pattern extending over one or more of the radially outer contact surfaces of the skin contact elements, wherein:
the surface pattern comprises a pattern of regular or irregular geometric features, or a combination thereof, adapted for frictional contact with an adjacent skin surface;
the surface pattern comprises a chevron or ridged pattern extending across the one or more contact surfaces, transverse to the rotational axis;
the surface pattern is adapted to stretch a portion of an adjacent skin surface, responsive to application of the force; and/or
the surface pattern is adapted to cleanse, exfoliate, or distribute a topical agent over an adjacent skin surface, responsive to application of the force.

19. The skin treatment device of claim 16, wherein:
the plurality of skin contact elements comprise two, four, six, eight, ten or twelve skin contact elements; or
the plurality of skin contact elements are distributed circumferentially about the axle or hub at substantially equal angles, or in a rotationally symmetric pattern about the rotational axis.

20. The skin treatment device of claim 16, further comprising:
a housing configured for supporting the axle or hub with one or more of the skin contact elements extending through a bottom surface thereof; or
a handle configured for rotational supporting the axle or hub with one or more of the skin contact elements extending therefrom;
wherein the radially outer contact surfaces of the one or more skin contact elements are adapted to receive the force via application to a skin surface under treatment.

21. The skin treatment device of claim 16, further comprising one or more conducting surfaces or electrodes adapted for applying a current treatment to a skin surface of a subject, wherein:
one or more of the conducting surfaces or electrodes are disposed in or on one or more of the radially outer skin contact surfaces;
one or more of the conducting surfaces or electrodes are interleaved or disposed between one or more adjacent pairs of the skin contact elements; and/or
one or more of the conducting surfaces or electrodes are configured for electrical contact with the skin surface upon application of the force, such that the respective longitudinal portions displace radially inward toward the one or more conducting surfaces or electrodes; and
further comprising one or more of a voltage source, a current source or a controller disposed in or on the device, and configured to provide a voltage or current waveform selected for application of the current treatment via the one or more conducting surfaces or electrodes.

22. The skin treatment device of claim 21, wherein:
one or more of the conducting surfaces or electrodes are disposed on a bottom surface of the device;
one or more of the conducting surfaces or electrodes comprise a patterned treatment surface extending over at least a portion of a bottom surface of the device; and/or
one or more of the conducting surfaces or electrodes are disposed on a handle of the device or on an upper surface of the device, spaced from a bottom surface thereof.

23. A non-transitory, machine readable data storage medium with program code stored thereon, the program code executable on a computer processor to apply a current treatment according to claim 21.

24. The skin treatment device of claim 16, wherein each of the skin contact elements defines a flexible compressive element comprising:
a first longitudinal section;
a second longitudinal section extending in parallel, spaced relation to the first longitudinal section; and
a transverse section comprising the one or more web elements extending between the first and second longitudinal sections at the angle thereto;
wherein the angle is a skew angle adapted for displacement of the second longitudinal section toward and along the first longitudinal section, in response to a load.

25. The skin treatment device of claim 24, wherein the skew angle is defined at or adjacent couplings to the first and second longitudinal members, each of the web elements having a medial portion extending between the couplings, and wherein:
a cross section of the web element is greater at or adjacent the couplings than in the medial portion; and/or
a flexibility of the web element is greater in the medial portion than at or adjacent the couplings.

26. The skin treatment device of claim 24, further comprising a contact surface on the second longitudinal section, opposite the first longitudinal section, wherein:
the contact surface has a ridged, scalloped, undulating, symmetric grid, spiral, radial, asymmetric, irregular, or randomized texture, extending transverse to a direction of the load;
the contact surface is adapted for frictional contact with an adjacent skin surface, via which the load is applied;
the contact surface is adapted to cleanse, exfoliate, or distribute a topical agent over an adjacent skin surface, responsive to the displacement along the first longitudinal section; and/or
the contact surface is adapted to stretch a portion of the skin surface between an adjacent pair of such flexible compressive elements, wherein the respective skew angles have an opposing orientation such that the displacements of the second longitudinal sections along the first longitudinal sections are in opposing directions in the adjacent pair.
